(19) United States
(12) Patent Application Publication
(10) Pub. No.: US 2008/0306407 A1
(43) Pub. Date: Dec. 11, 2008

(54) Bandage Pressure Sensor

(75) Inventor: Michael Taylor, Leafield (GB)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: PRESSURE MEDICAL LTD., Leafield (GB)

(21) Appl. No.: 11/910,394

(22) PCT Filed: Mar. 29, 2006
(86) PCT No.: PCT/GB2006/001137

§ 371 Date: Jun. 24, 2008

(30) Foreign Application Priority Data
Mar. 29, 2005 (GB) 0506308.6

(51) Int. Cl. A61B 5/103; A61L 15/00; A61B 19/00

(52) U.S. Cl. 600/587; 602/75; 128/898

(57) ABSTRACT

A pressure sensor for providing an indication of the pressure applied by a bandage to a human or animal body. The sensor comprises an elongate, flexible support strip (63) adapted to be placed between a bandage and the body, the support strip carrying a flat pressure sensitive portion (69) whose electrical properties vary with applied pressure perpendicular to the general plane of the support strip, and also carrying flat, flexible conductors (65, 66) for connecting the pressure sensitive portion to a supply of electricity and to means for providing an indication of the applied pressure (71). In some arrangements an integrated battery, circuitry and display may be provided on the support strip.

This invention relates to apparatus for monitoring the pressure of a bandage applied to a human or animal body, for example to hold a dressing in place or to act as a support.

The expression “bandage” used herein encompasses bandages which must be wound round a body part, as well as tubular elasticated bandages, support hosiery, adhesive plasters, plaster casts, resin casts, and so forth.

Wound therapy, such as for the treatment of leg ulcers, requires the application of bandages with a degree of pressure. It is known that excessive pressure when applying a bandage can cause discomfort or harm to a patient. Similarly, insufficient pressure may provide insufficient support or may fail to locate a dressing in place properly. The judgement of the correct pressure requires experience and skill on the part of the person applying the bandage. Whilst experienced nurses may be able to judge the correct pressure reasonably well, there is still room for error.

Systems have been developed to assist in judging the correct pressure. For example, it is known to provide an elastic bandage with shapes which expand as the bandage is stretched whilst being wound round e.g. a leg or other part of a patient. Once the shape has expanded to a predetermined form, such as a square or circle, then it is assumed that an appropriate pressure has been achieved. One such arrangement is disclosed in EP0597749. This is an inaccurate means of measuring pressure, and is also dependent on using the appropriate bandage. There is still a risk of discomfort or harm if this system is used by a less skilled or experienced person.

More accurate means of pressure measurement exist in various fields, but they are generally inappropriate for use in monitoring the pressure applied by a bandage to a patient. Issues such as expense, disposability, ease of use by less skilled workers, and patient comfort are important as well as accuracy of measurement. WO-A-9718450 discloses a system for measuring the pressure on a foot or a horse's hoof, for example, which is unsuitable for measuring the pressure applied by a bandage.

GB-A-2199953 discloses an arrangement for measuring pressure on the skin of a bed bound patient or under a compression bandage. It uses a fluid filled cell which is connected to a piezo electric sensor by a fluid filled tube, or which is inflated to close contacts. Such an arrangement is complex and relatively bulky. U.S. Pat. No. 5,838,244 discloses another arrangement with an inflatable sensor. DE-20119785-U1 discloses a pressure measurement system for use on a horse, which is bulky. DE-A-3132557 and U.S. Pat. No. 4,858,620 also disclose arrangements which are bulky.

Viewed from one aspect of the present invention, there is provided a pressure sensor for providing an indication of the pressure applied by a bandage to a human or animal body, the sensor comprising an elongate, flexible support strip adapted to be placed between a bandage and the body, the support strip carrying a flat pressure sensitive portion whose electrical properties vary with applied pressure perpendicular to the general plane of the support strip, and also carrying flat, flexible conductors for connecting the pressure sensitive portion to a supply of electricity and to means for providing an indication of the applied pressure.

In use the support may be placed directly on the body, but for reasons of comfort and hygiene it is preferably placed between the windings of the bandage, or between the bandage and a sleeve—such as an elastic “stockingette”—worn by the patient. For example, a nurse or other carer may form a few windings of a bandage, position the support and then make some windings of the bandage over the support to ensure that it is correctly positioned, before carrying on bandaging in the normal way. Part of the support, or a connection to the support, preferably protrudes through the windings for various purposes. In the case of a monitor that is completely self contained, the protruding part could carry e.g. an activating switch and/or a display. For a monitor that uses an external source of power and/or an external display, connections only could protrude.

In one preferred arrangement, the pressure sensitive portion is provided adjacent one end of the elongate support strip and electrical terminals are provided adjacent the other end of the strip. In another preferred arrangement, with an integral display, the pressure sensitive portion is provided adjacent one end of the elongate support strip and display is provided adjacent the other end of the strip.

One advantage of having the support protrude from the bandage is that if desired it can be slid out after use, to reduce the risk of discomfort to the patient. Because the support is flat and flexible, in certain embodiments it can be withdrawn with little disruption to the bandaging or effect on the pressure subsequently.

Preferably, in addition to the support being flat, anything else covered by windings of a bandage, such as separate connecting leads, are also flat and flexible. This is to avoid discomfort to a patient. In the preferred embodiment, such connecting leads are provided integrally on the support, which in use is arranged to project beyond or between windings of a bandage so as to be accessible. However, it would be possible to have the support completely covered by a bandage, with leads extending along and protruding from the bandage.

Preferably, the pressure sensitive portion itself is flexible and in a preferred arrangement both the support strip and the pressure sensitive portion can be adapted to the shape of a body part by flexing about both longitudinal and lateral axes with respect to the elongate support strip. Preferably the pressure sensitive portion is a membrane. The membrane may have thickness of less than about 200 microns, and may be between about 50 microns and 150 microns, with possible ranges being 100 to 150 microns or 50 to 100 microns, or 125 microns to 175 microns. One suitable material could be a composite made from a polymer filled with carbon. In these some carbon particles always contact one another creating a conduction path. As pressure is applied, more come into contact and therefore more conduction pathways build up. This conduction process is known as percolation. However, the preferred material is a Quantum Tunnelling Composite (QTC), which is available in flexible form as sheets and other products—and might be obtained as a printable ink—and whose conductivity changes in accordance with pressure applied across the plane of the sheet. In general, the conductivity increases as the pressure increases. In QTC's the conduction process is fundamentally different to that of conventional carbon filled polymer sensors. In QTC's the conductive, metal, particles in the polymer never come into contact. They do however get very close as pressure increases, so close that quantum tunnelling is possible between the metal particles to establish conductive paths.

A small portion only of the QTC sensor material will generally be required in preferred embodiments of the invention. However, it should be appreciated that other pressure sensitive portions may be used, such as the carbon filled polymers mentioned above, capacitive sensors, piezo-electric sensors and so forth. QTC's are preferred as they have been considerably developed, are relatively simple to utilise and can be very thin and flexible. In general, a flat, thin, flexible pressure sensitive material is preferred for use in embodiments of the present invention.

The support may be in the form of a sheet or other element, but in the preferred embodiment is elongate and in the form of a strip. This reduces the overall size and also the potential for discomfort to a patient.

The support is preferably of the minimum functional thickness, for example ranging from about 1 mm to paper thin, to reduce the possibility of discomfort to a patient. Depending on the material of the substrate, 1 mm may be too thick to provide full flexibility, and around 0.25 mm to 0.5 mm may be a maximum thickness. Typically, the substrate may be around 100 μ thick, and the pressure sensitive portion may have a thickness of, for example, less than 200 μ, and may be between about 50 and 150 μ, with possible ranges being 100 to 150 μ or 50 to 100 μ, or 125 to 175 μ.

The means for providing an indication of the applied pressure in accordance with the electrical properties of the pressure sensitive portion, could be part of an external unit connected to the pressure monitor. In such a case, connectors such as sockets could be provided on the support, for plugs/wires to be attached to the external unit. The external unit could also contain a power supply, either a battery or a safe, transformed supply from the mains electricity. The external unit could also be provided with controls for the system, such as an on/off switch or a calibration sequence. The indication for the applied pressure could be audio, visual or both. There could be a real or virtual needle, a bar display and so forth. In a preferred arrangement there is a visual arrangement which requires little technical skill on the part of the user, such as different colour segments which are illuminated in accordance with the applied pressure. There could, for example, be one colour to indicate an acceptable range of pressure, and at least one other colour to indicate excessively high or excessively low pressures. Alternatively, a monochromatic display could be used, in which segments progressively change colour as the pressure increases. This could be provided by a liquid crystal display, which can be both thin and flexible and could thus be incorporated on a flexible support strip.

In practice, an external unit could be made very compact, for example being no bigger than a pen, watch or the like, and thus easily portable by a nurse or other carer. In one preferred arrangement, however, the monitor is entirely self contained, with its own battery, display and e.g. control switch on the support. Items on a portion of the support which protrudes from the windings of a bandage in use, could be less of a flat nature than the main part of the support. Thus, a battery, display and control switch could be provided on this portion, connected to the pressure sensitive portion, preferably by means of flat conductors. Preferably, however, all components are kept relatively flat. Thus a battery could be printed, as well as connectors and a display. A printed battery is available under the Trade Mark “Power Paper” from Graphic Solutions in the United States of America.

The display could be a thermo-chromic element that changes colour with increases in current, or there could be a coloured or monochromatic series of elements that become illuminated as current increases or decreases, providing a variable scale. Other circuit elements such as an integrated circuit chip programmed with suitable logic can also be kept small in size. In the case of a printed battery, power output may be an issue and, for example, the display may need to have a low power consumption—for example being a monochrome LCD display rather than use coloured LED's.

A monitor of this type could be designed to be disposable after a single use or a limited number of uses, as could a monitor requiring an external unit. Disposal prevents the risk of cross infection, a major advantage. However, if kept for use on a single patient, multiple use might be acceptable. A support of a suitable thin, plastics material provided with a pair of conductors and a QTC pressure sensitive portion laminated in position, for example, can be made very inexpensively. The conductors could be applied to the main substrate of the support, such as a plastics strip, by the application of metallic wires or strips, foil blocking with a conductive foil, printing with conductive ink, or by any other suitable technique. A portion of a QTC pressure sensitive sheet is engaged with the conductors, and may be held in pace under a small degree of compression, for example by means of a layer attached to the support. This could be performed during lamination. In some circumstances the pressure sensitive sheet could be conductively bonded to the conductors. Even with an integral battery and pressure indicator display, the cost of the support can be appropriate for disposal after single use.

Monitors which require an external control unit for power/display functions can be manufactured very inexpensively. This may be of particular advantage in situations where it is desired to monitor pressure at a number of points in the region being bandaged. The outputs from a number of monitoring strips or the like can be fed to a multi channel monitoring unit, with separate pressure indicators for the different monitors. For example, there could be three channels for three monitors placed adjacent the ankle, calf and knee of a patient with extensive bandaging over the whole of the lower leg. Although three sensors, in this particular example, are required, their inexpensive construction makes monitoring at multiple sites feasible even when they have an integral battery and display. The number of sites chosen will depend on the particular application concerned.

It will be appreciated that there are many variations possible and, for example, the individual sensors could have integral displays but be connected to a common power source, or even have integral power sources and be connected to a common multi-channel control unit for display purposes. There could be a single type of sensor which has an integral display and can be used as a self contained unit, but is also connectible to an external unit. Thus, if a particular scenario requires multiple site monitoring, the same type of monitor can be used but the displays can be viewed centrally, thus making it easier for the nurse or other carer to view the pressure characteristics at the different sites.

Where multi site monitoring is required, a multi channel unit can display all readings simultaneously, or cycle through the readings from individual monitors. There could be a display of which monitor's reading is being displayed, for example numerically or descriptively, such as “top”, “middle”, bottom”.

The monitors, when in the form of elongate strips, may be provided in a selection of lengths. For example, if “top”, “middle” and bottom” monitors are employed they could be of increasing length so that they all protrude from the bandage at about the same point. This means that they can easily be connected to a common cable for feeding to a control unit.

The support may be somewhat adhesive, for example having an adhesive layer or having hooks (of the type found on hook and loop fastening strips) which will provide some adhesion to a bandage or to a sleeve worn by the patient. This will assist in positioning the monitors correctly whilst bandaging takes place. However, such a support cannot normally be slid out of position after use. Different types of support can be provided for different situations and user preferences.

The circuitry provided, either embedded on the support itself or in a separate control unit, preferably smoothes/averages pressure readings over time to avoid undesirable fluctuations and “chattering” of display elements.

Typically, the monitor may be arranged to provide displays equivalent to pressures in the range from about 10 mm Hg to 125 mm Hg. A typical range for displayed pressures may be from about 10 mm Hg (too little pressure) to 60 mm Hg (too much pressure), with an acceptable pressure being about 40 mm Hg. In practice the figures as such may or may not be displayed, and instead appropriate positions on a bar or appropriate colours may be used to indicate acceptable and non-acceptable pressures.

It will be appreciated that where an external control unit is provided, this could be adapted not only to provide an indication of correct pressure during application of the bandage, but also to monitor pressure changes continually. Data storage in the form of e.g. solid state memory could be provided, and logic/output devices/ports provided to provide reports. An alarm could be provided to give an indication of an unacceptable pressure change, for example if a bandage starts to unravel or if pressure rises unexpectedly.

The invention also extends to a package comprising a bandage and one or more, preferably single use/disposable, monitors as above described.

Another aspect of the invention provides a method of providing an indication of the pressure applied by a bandage to a human or animal body, the method comprising the step of positioning a sensor between a bandage and the body, the sensor comprising an elongate, flexible support strip carrying a flat pressure sensitive portion whose electrical properties vary with applied pressure perpendicular to the general plane of the support strip, and also carrying flat, flexible conductors which connect the pressure sensitive portion to a supply of electricity and to means for providing an indication of the applied pressure.

Another aspect provides a method of applying a bandage to a human or animal body, in which there is provided an indication of the pressure applied by the bandage to the human or animal body and the bandage is applied in such a way that the applied pressure is kept within predetermined acceptable limits, the method comprising the step of positioning a sensor between a bandage and the body, the sensor comprising an elongate, flexible support strip carrying a flat pressure sensitive portion whose electrical properties vary with applied pressure perpendicular to the general plane of the support strip, and also carrying flat, flexible conductors which connect the pressure sensitive portion to a supply of electricity and to means for providing an indication of the applied pressure.

Viewed from another aspect of the present invention, there is provided a pressure sensor for providing an indication of the pressure applied by a bandage to a human or animal body, the sensor comprising a flat, flexible support adapted to be placed between a bandage and the body, the support being provided with a pressure sensitive portion whose electrical properties vary with applied pressure perpendicular to the general plane of the support, and with means for connecting the pressure sensitive portion to a supply of electricity and to means for providing an indication of the applied pressure in accordance with the electrical properties of the pressure sensitive portion.

Whilst in the above aspects of the invention, the substrate is flat, thin and elongate, it will be appreciated that other aspects of the invention extend to the various components used in other scenarios. For example, a flat pressure sensor could be provided with separate leads going to a control and/or display unit. Whilst less convenient for use, the results could be the same.

Accordingly, another aspect of the invention provides a method of providing an indication of the pressure applied by a bandage to a human or animal body, the method comprising the step of positioning a sensor between a bandage and the body, the sensor comprising a flat, flexible support carrying a flat, flexible pressure sensitive portion whose electrical properties vary with applied pressure perpendicular to the general plane of the support strip, and also carrying flat, flexible conductors which connect the pressure sensitive portion to a supply of electricity and to means for providing an indication of the applied pressure.

Viewed from another aspect, the invention provides a pressure sensor for providing an indication of the pressure applied by a bandage to a human or animal body, the sensor comprising an flexible support substrate adapted to be placed between a bandage and the body, the support substrate carrying a flat pressure sensitive portion in the form of a flexible membrane whose electrical properties vary with applied pressure perpendicular to the plane of the membrane, and also carrying flat, flexible conductors for connecting the pressure sensitive portion to a supply of electricity and to means for providing an indication of the applied pressure. The membrane may have a thickness of less than 200 microns.

Viewed from another aspect, the invention provides a pressure sensor for providing an indication of the pressure applied by a bandage to a human or animal body, the sensor comprising an flexible support substrate adapted to be placed between a bandage and the body, the support substrate carrying (a) a flat pressure sensitive portion whose electrical properties vary with applied pressure perpendicular to the plane of the substrate; (b) a battery; (c) means for providing an indication of the applied pressure; and (d) flexible conductors which connect the pressure sensitive portion to the supply of electricity and to the means for providing an indication of the applied pressure. The battery is preferably low profile, for example being a printed battery.

According to another aspect of the invention, there is provided a method of providing an indication of the pressure applied by a bandage to a human or animal body, the method comprising the step of positioning a sensor between a bandage and the body, the sensor having a thickness of no more than 0.5 mm and comprising a flexible substrate, flexible conductors provided on the substrate, and a pressure sensitive portion in contact with the flexible conductors, the pressure sensitive portion being of a material whose electrical properties vary with applied pressure perpendicular to the general plane of the substrate.

In addition to use when a bandage is applied to an animal or human body, a sensor in accordance with the invention may be used to measure the pressure when a bandage is applied to a test part of the body, for example to evaluate the properties of a bandage—for example applying hosiery to an imitation leg—or in a training exercise.

Some embodiments of the invention will now be described by way of example and with reference to the accompanying drawings, in which:-

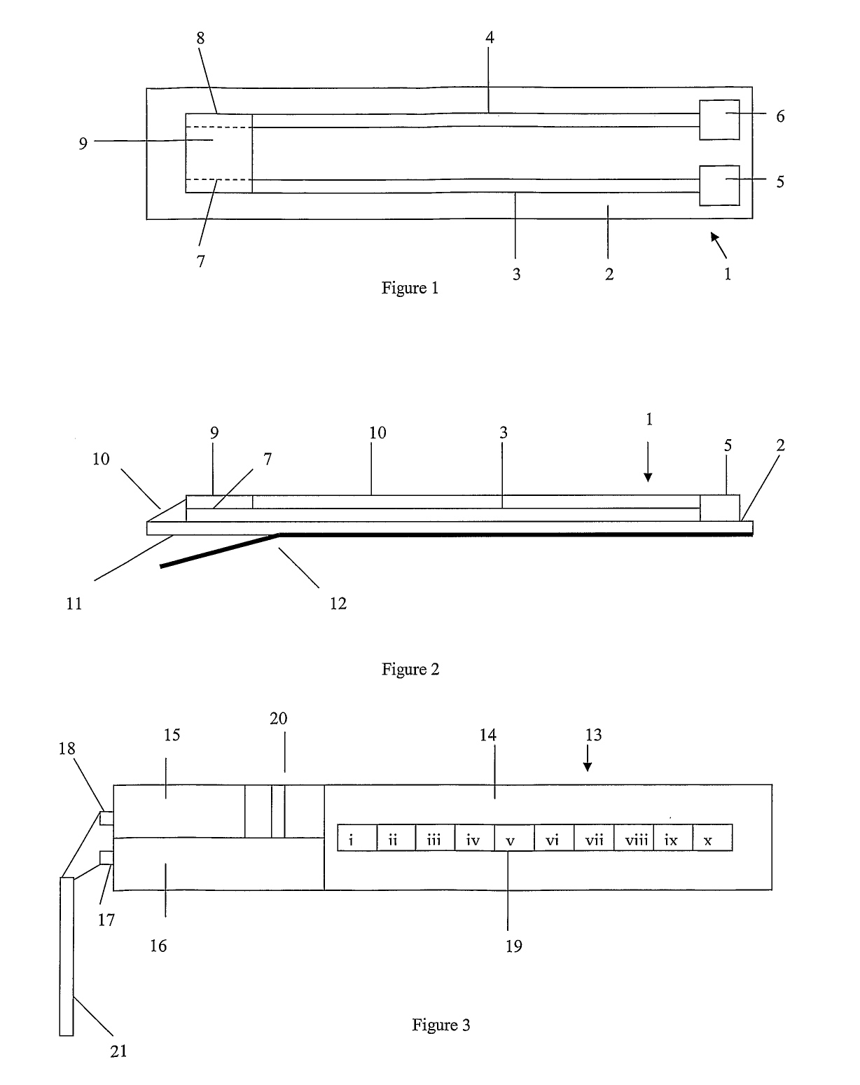

FIG. 1 is a plan view of a first embodiment of a pressure monitor in accordance with the invention;

FIG. 2 is a side view of the monitor;

FIG. 3 is a view of a control unit used with the monitor;

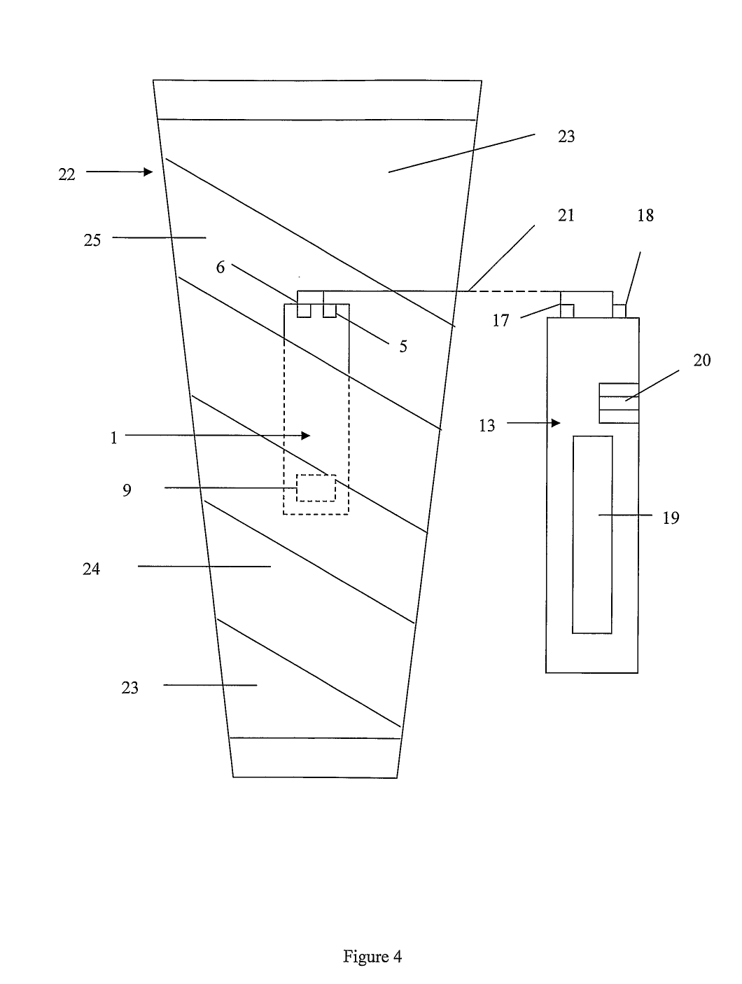

FIG. 4 is a view of the monitor in use;

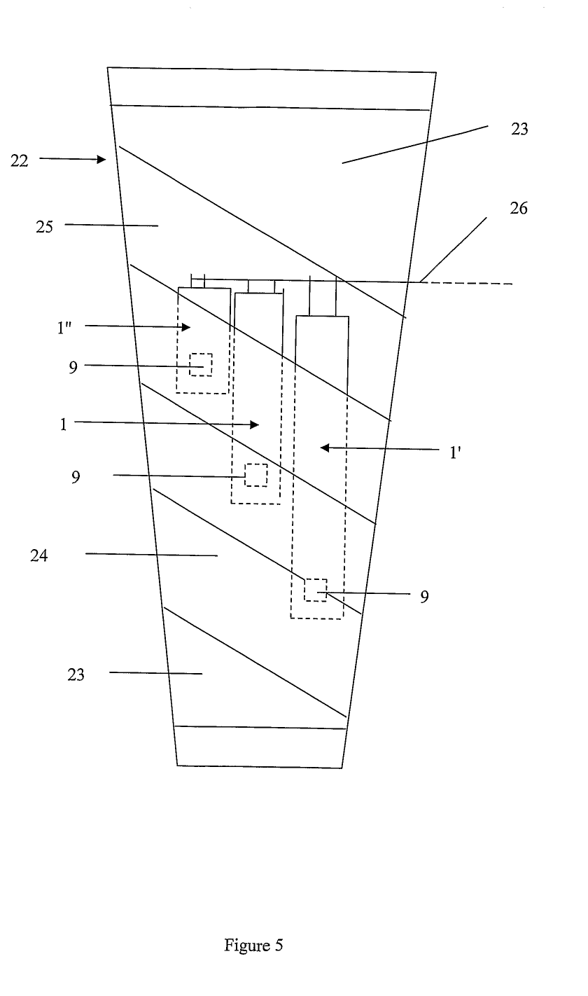

FIG. 5 is a view showing a number of the monitors in use;

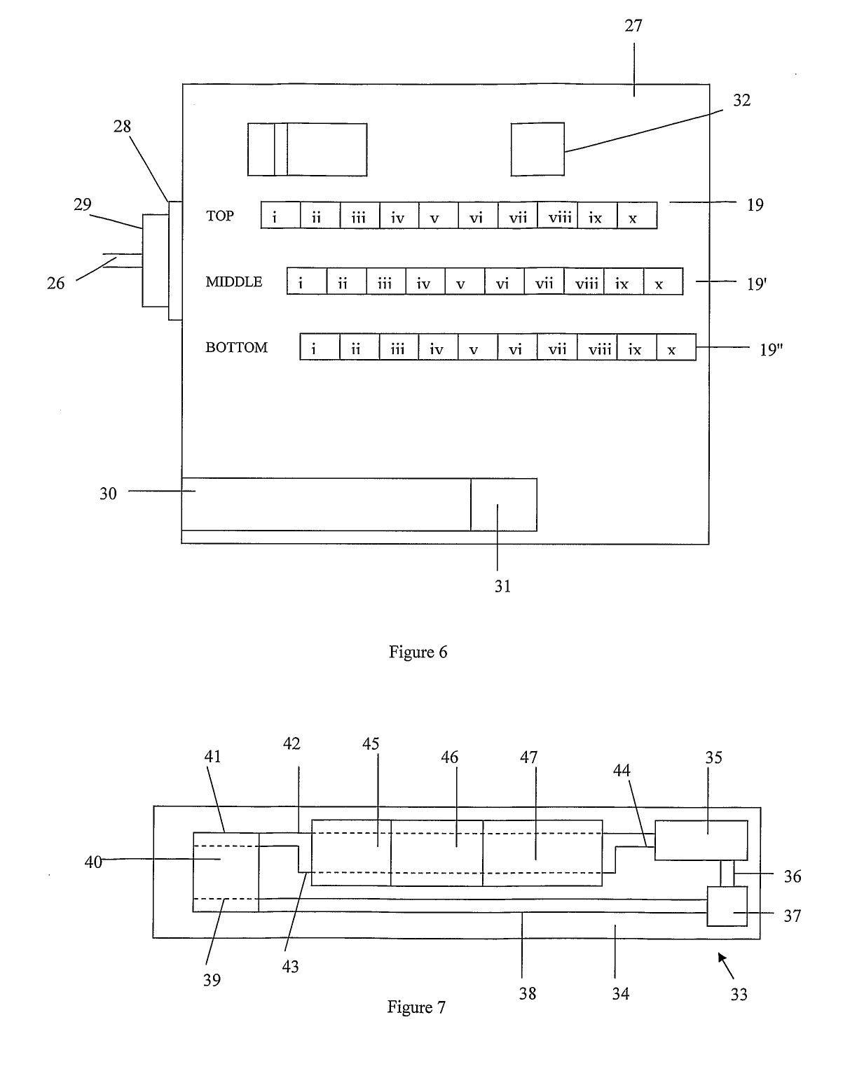

FIG. 6 is a view of an alternative control unit for use with the monitors in the arrangement of FIG. 5;

FIG. 7 is a plan view of a second embodiment of a pressure monitor in accordance with the invention;

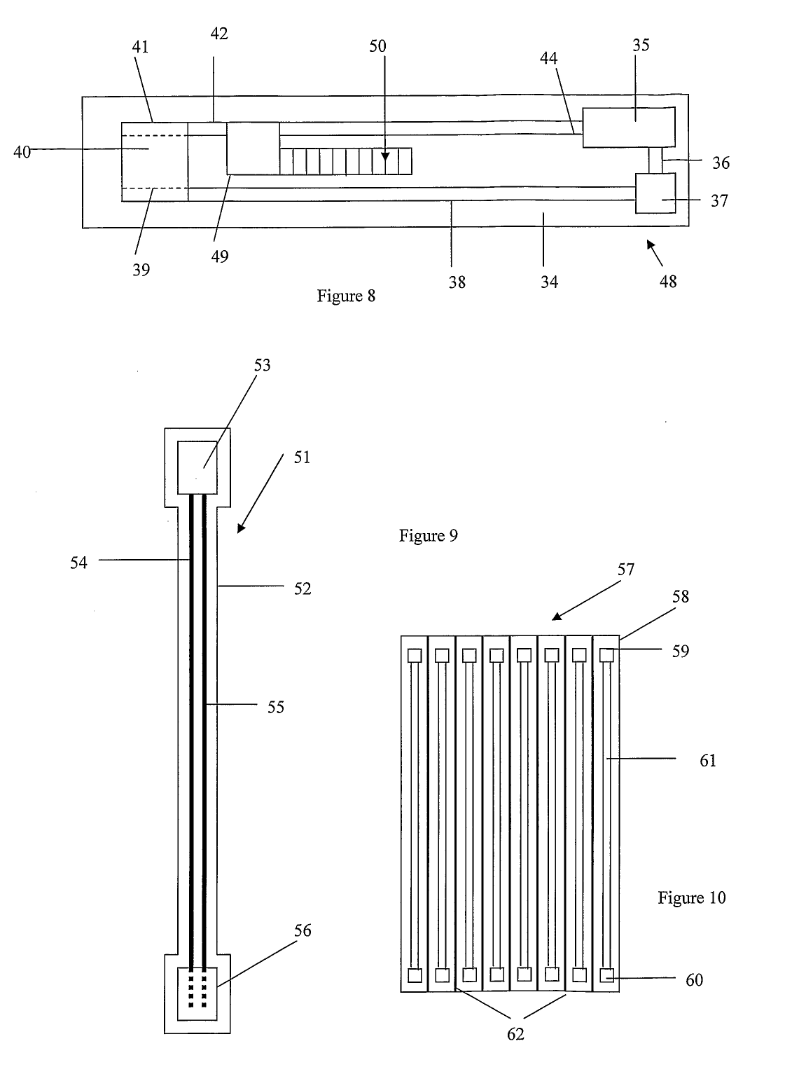

FIG. 8 is a plan view of a third embodiment;

FIG. 9 is a plan view of a fourth embodiment; and

FIG. 10 is a plan view showing a possible manufacturing process.

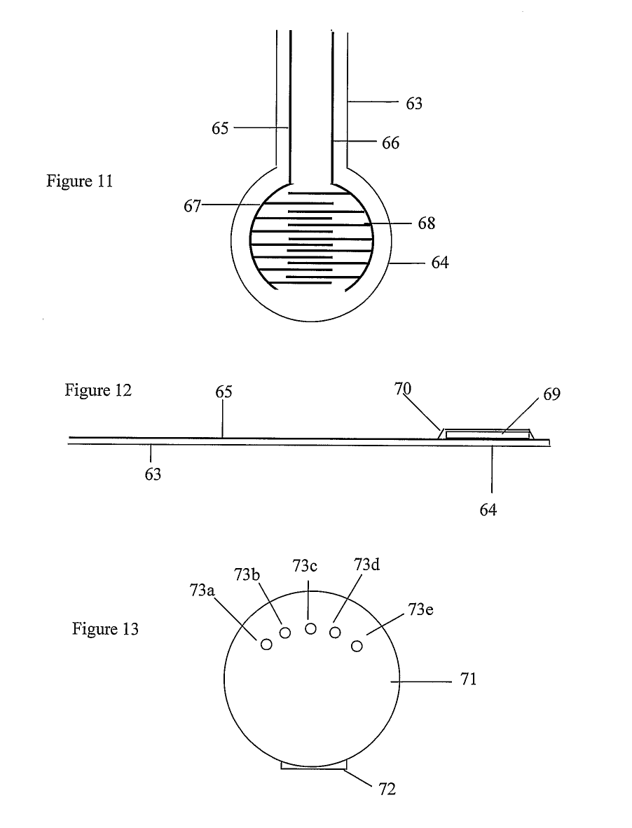

FIG. 11 is a plan view of one end of another embodiment of sensor;

FIG. 12 is a diagrammatic side view of the sensor of FIG. 11; and

FIG. 13 shows an embodiment of a processing and display unit.

As shown in FIGS. 1 and 2, a monitor 1 comprises a flat, thin, flexible substrate 2 of plastics or other suitable material such as card. In this embodiment the construction is such that the substrate is electrically insulating. Adhered to the substrate are two thin, flat, flexible conducting foil tracks 3 and 4. At one end, these terminate in electrical connectors 5 and 6 bonded to the substrate 2. At the other end, portions 7 and 8 of the tracks are bridged by a small portion 9 of a thin, flat, flexible pressure sensitive material. In this embodiment, this is a portion cut from a sheet of a pressure sensitive quantum tunnelling composite (QTC) of the type available for example from Peratech Ltd, Darlington, County Durham, England. The portion 9 is pressed into conductive contact with the end portions 7 and 8 of the tracks 3 and 4 by a flexible insulating laminating layer 10, e.g. of plastics or paper which also covers the substrate 2 and tracks 3 and 4, and is bonded to the substrate 2, the portion 9 and the tracks 3 and 4. On its underside the substrate is provided with a weakly adhesive layer 11 covered by an optional removable cover strip 12, shown partly removed in FIG. 2.

It will be appreciated that in FIG. 2 in particular, the scale is greatly exaggerated to show the components and that in practice the complete monitor 1 is flat, thin and flexible.

FIG. 3 shows a control unit 13 comprising a small, pen like housing 14 provided with a battery 15, control circuitry 16, connectors 17 and 18, a display 19, and an on/off switch 20. In use the control unit 13 is connected to the monitor 1 by twin wire lead (e.g. twisted pair) 21 so that connector 5 is connected electrically to connector 17 and connector 6 is connected to connector 18. When switch 12 is activated to the on position, the battery 15, control circuitry, track 3, QTC portion 9, and track 4 are connected in series. In an alternative arrangement, there could be no switch as such and the unit 13 could be activated automatically when connected to the monitor 1. As the pressure on the QTC portion 9 is varied, the electrical conductivity varies, and the conductivity is detected by the control circuitry 16. This control circuitry includes means for damping or averaging out readings over time, so as to avoid constant fluctuations in output from the control circuitry and also to compensate for any hysteresis of the sensor material. This output, dependent on the conductivity detected and thus the pressure on the QTC portion 9, drives the display 19. As shown, the display 19 comprises a linear series of light emitting diodes (LED's) of different colours. In this particular example there are ten LED's (i to x)which are arranged to light up progressively corresponding to 5 mm Hg pressure increments, from 10 mm Hg (i) to 60 mm Hg and above (x). In this particular example, they are colour coded to assist in detecting the appropriate pressure. For example, LED's i and ii could be yellow, iii and iv light blue, v, vi and vii green, viii dark blue, ix purple and x red. The green LED's indicate the correct pressure range, whilst LED's i to iv indicate insufficient pressure and LED's viii to x indicate excessive pressure. It will be appreciated that many other types of display, both coloured and monochromatic, could be employed and this is by way of an example only.

FIG. 4 shows the monitor 1 and the control unit 13 in use during the bandaging of the limb 22 of a patient. The limb is clad with an elasticated stockingette layer 23 to improve the comfort of the patient. The strip 12 has been removed from the sensor 1, whose adhesive surface 11 is adhered to the layer 23 at a suitable position. An elasticated bandage 24 is wound around the limb, over the layer 23 and the sensor 1. The arrangement is such that the pressure sensitive portion 9 of the sensor is covered by the bandage 24. Towards the upper end, the winding 25 of the bandage passes behind the sensor 1, whose upper end protrudes to reveal connectors 5 and 6. These are connected by lead 21 to connectors 17 and 18 on the control unit 13. The switch 20 is turned to the on position whilst the bandage is wound around the limb, and the display 19 indicates the pressure detected by the sensor, in accordance with the pressure on the portion 9. The nurse or other carer watches the display whilst the bandage is wound, and adjusts the tension on the bandage to ensure that the pressure is in the correct range as indicated by the display 19.

After bandaging is completed, the lead 21 is disconnected from the monitor 1 and the control unit 1 switched off. In this embodiment the sensor 1 is left in place, but being thin, flat and flexible it does not cause discomfort to the patient. When a fresh bandage is required, the existing bandage is unwound and the sensor 1 and/or bandage are re-used or disposed of hygienically. In general, a fresh bandage with be used either with a re-used sensor or preferably a new sensor, in conjunction with control unit 13, in the manner described above.

FIG. 5 shows an arrangement using three sensors 1, 1′ and 1″ to monitor pressure at three points on the leg. Three identical sensors 1 could be used, protruding from the bandage 24 at different heights, or at the same height but arranged at different portions around the leg. In this particular embodiment, the sensors protrude at about the same height, but are of different lengths. Sensors 1′ and 1″ are identical in construction to sensor 1, with the exception that sensor 1′ is longer, with longer conductive tracks from the sensor portions 9 to the connectors at the upper ends, and sensor 1″ is shorter. The sensor portions 9 are at three different heights. A cable 26, with three pairs of wires, is used to connect the three sensors to a special multi channel control unit 27 as shown in

FIG. 6, the cable having three pairs of connectors at the monitor end for connection to the three pairs of connectors 5, 6 that there will be.

FIG. 6 shows the control unit 27 for use with the system of FIG. 5. In terms of construction the control unit is similar to that 13 described earlier, but it is somewhat larger. Instead of connectors 17 and 18 for cable 21, there is a multi channel socket 28 for receiving a multi channel plug 29 on the end of cable 26. There are three bar displays, 19, 19′, 19″ representing the pressure at the three points being monitored.

This particular control unit also has a pressure data storage area 30 and a port 31 so that the stored data can be accessed. The port can be wired for linking to suitable apparatus, for example being of the Universal Serial Bus (USB) type or wireless, such as a Bluetooth transceiver. It also has a warning light and/or audible alarm 32, if the pressure increases or decreases by an unacceptable amount whilst the pressure is being monitored over an extended period. This feature can be enabled by the carer after the bandage has been positioned, so as not to produce alarms whilst bandaging is taking place. However, audible indicators can optionally be provided to supplement or replace visible displays in embodiments of the invention.

FIG. 7 is a plan view of an alternative monitor 33 which does not need a separate control unit, and is primarily intended for single use only. The monitor 33 comprises a flat, thin, flexible substrate 34 of plastics or other suitable material such as card. In this embodiment the material is such that the substrate is electrically insulating. Printed or otherwise provided as flat, thin flexible objects are a number of electrical components. One terminal of a battery 35 is connected by an electrically conducting strip 36 to a switch 37, and then to an elongate conductive track 38 terminating in a portion 39. Laterally spaced from this is a portion 41 of another conductive track 42. Portions 41 and 39 of the tracks are bridged by a small portion 40 of a thin, flat, flexible pressure sensitive material. In this embodiment, this is a portion cut from a sheet of a pressure sensitive quantum tunnelling composite (QTC) of the type available for example from Peratech Ltd, Darlington, County Durham, England. The portion 40 is pressed into conductive contact with the end portions 39 and 41 of the tracks 38 and 42 by a flexible plastics insulating laminating layer, not shown. A conductive grease could be used between the portion 40 and the track end portions 39 and 41.

Track 42 is connected to another track 44, in turn connected to the other terminal of battery 35, by a resistive element 43. Over this are positioned three portions of thermochromic material 45, 46 and 47. These are designed to change to different colours, and at different temperatures. For example, from a base colour portion 45 may change to yellow at one temperature, portion 46 to green at a higher temperature, and portion 47 to red at a higher temperature still. These temperatures are however chosen to be relatively low, so as not to harm a patient. The laminating layer covers all of the components, to protect them. The underside of the substrate may be provided with an adhesive layer, optionally with a release paper if necessary, as in the first embodiment.

In use, when the switch is activated, the current flowing depends on the conductivity of portion 40. As the pressure increases, the current increases and the temperature of the resistive element 43 increases. The colours of the thermochromic portions 45, 46 and 47 alter in accordance with the pressure. In this particular embodiment, the central portion 46 changing to green indicates the correct pressure, and if portion 47 changes to red the pressure is too high. In practice, for reason of simplicity, the colour ranges may be more basic and the effect may be similar to the type of strip thermometer that can be placed on a person's forehead.

In an alternative arrangement as shown in FIG. 8, the monitor 48 is of similar construction but in place of the resistive element 43 and thermochromic portions, there is provided a control unit 49 and a display 50, similar to those in the control unit 13 of the first embodiment, but made sufficiently thin, flat and flexible to be on the support itself. The display 50 may be monochrome for reasons of simplicity and expense, and may act essentially like a bar thermometer.

For the purposes of illustrating features of the embodiments clearly, they are not shown to scale in the figures referred to above. In practice, they could be considerably more elongate. FIG. 9 is a plan view of a further embodiment of a monitor 51, shown as being more elongated. The monitor comprises a long, flat, thin substrate 52 with an enlarged upper end on which is mounted circuitry 53, for example including a battery, switch, processing circuitry and a display. This is connected by printed tracks 54 and 55 to a thin portion of QTC pressure sensitive material on an enlarged lower end of the monitor. The monitor can function in the same manner as any of the monitors described earlier.

Referring now to FIG. 10, there is shown a sheet 57 of thin, flat, flexible plastics substrate material. On this are defined a number of elongate monitors 58, each having thereon control circuitry 59 at one end, a pressure sensitive portion 60 at the other end, and an interconnecting conductive track pair 61. After the portions have been printed, bonded or otherwise connected to the substrate sheet in this parallel formation, the individual monitors can be separated by cutting along lines 62.

FIG. 11 shows one end of an alternative sensor, comprising an elongate thin, flexible support strip 63 terminating in a circular end portion 64. The support strip carries flat, flexible conductors 65 and 66 which terminate in interlaced fingers 67 and 68 respectively on the end portion 64. As shown in FIG. 12, a QTC pressure sensitive portion 69 is laid over the fingers 67, 68, and is held in place by a sheet of material 70 bonded to the support strip 63. In this case, the portion 69 is about 10 mm square and 0.06 mm thick. The support strip 63 comprises a polyester substrate of e.g. 0.11 mm thick and there is a protective paper cover of 0.15 mm thick, giving a total thickness of around 0.27 mm.

Typically, the main part of the support strip 63 could be about 5 mm to 10 mm wide and the end portion 64 could have a diameter of about 20 mm. The total length of the sensor could be from about 100 mm to about 250 mm.

In a modification, the pressure sensitive portion 69 could be surrounded by a frame of the same thickness as the portion 69, the sheet 70 then covering both the portion 69 and the frame.

In an alternative arrangement, there could be a ribbon cable which has a portion of insulating material removed at one end to expose wires which can then be used as electrodes in contact with the QTC potion.

As shown in FIG. 13, a display module 71 is provided with a zero insertion force (“ZIF”) 72 socket which can, for example, receive directly the ends of conductors 65 and 66. The module 71 contains a battery and processing and display circuitry which drives 5 coloured LED's 73a to 73e. LED's 73a and 73e are red and are displayed when the pressure is respectively too little or too great by a critical amount. LED's 73b and 73d are yellow and are displayed when the pressure is respectively too little or too great, but not by a critical amount. LED 73c is green and is displayed when the pressure is correct. The operating voltage may be 1.5 volts, and the maximum current drain at fall pressure is about 10 μamps. The module 71 includes a data logging memory, and data can be transferred to a computer by means of a wired connection such as Universal Serial Bus (USB), or a wireless connection such as Bluetooth or another wireless communications protocol. It would also be possible to modulate one or more of the LED's to transmit data.

In embodiments of the invention, the pressure sensitive portion is preferably a QTC membrane, such as a switch substrate available from Peratech, which may have thickness of less than about 200 microns, and may be between about 50 microns and 150 microns, with possible ranges being less than 100 microns, or 100 to 150 microns or 50 to 100 microns.

1. A pressure sensor for providing an indication of the pressure applied by a bandage to a human or animal body, the sensor comprising an elongate, flexible support strip adapted to be placed between a bandage and the body, the support strip carrying a flat pressure sensitive portion whose electrical properties vary with applied pressure perpendicular to the general plane of the support strip, and also carrying flat, flexible conductors for connecting the pressure sensitive portion to a supply of electricity and to means for providing an indication of the applied pressure.

2. A pressure sensor as claimed in claim 1, wherein the support strip carries terminals connected to the flexible conductors.

3. A pressure sensor as claimed in claim 2, wherein the terminals are enlarged end portions of the conductors.

4. A pressure sensor as claimed in claim 2, wherein the terminals are adjacent one end of the support strip and the pressure sensitive portion is adjacent the other end of the support strip.

5. A pressure sensor as claimed in claim 2, in combination with a display module which includes a supply of electricity and means for providing an indication of the applied pressure, the display module being connected to the conductors.

6. A pressure sensor as claimed in claim 1, wherein the support strip carries a battery and means for providing an indication of the applied pressure, connected to the conductors.

7. A pressure sensor as claimed in claim 5, wherein the means for providing an indication of the applied pressure includes a visual display adjacent one end of the support strip and the pressure sensitive portion is adjacent the other end of the support strip.

8. A pressure sensor as claimed claim 1, wherein the support strip can be flexed about both longitudinal and transverse axes.

9. A pressure sensor as claimed in claim 1, wherein the pressure sensitive portion is flexible.

10. A pressure sensor as claimed in claim 9, wherein the pressure sensitive portion can be flexed about both longitudinal and transverse axes.

11. A pressure sensor as claimed in claim 1, wherein the pressure sensitive portion is of a material whose conductivity changes with applied pressure.

12. A pressure sensor as claimed in claim 11, wherein the pressure sensitive portion is of a quantum tunnelling composite.

13. A pressure sensor as claimed in claim 1, wherein the thickness of the sensor is no more that 1 mm.

14. A pressure sensor as claimed in claim 13, wherein the thickness of the sensor is no more that 0.5 mm.

15. A pressure sensor as claimed in any preceding claim 1, wherein the pressure sensitive portion has a thickness of less than about 200 microns.

16. A pressure sensor as claimed in claim 15, wherein the pressure sensitive portion has a thickness of between about 125 and 175 microns.

17. A pressure sensor as claimed in claim 15, wherein the pressure sensitive portion has a thickness of less than 100 microns.

18. A pressure sensor as claimed in claim 1, wherein the pressure sensitive portion is a membrane.

19. A pressure sensor as claimed in claim 1, wherein the support strip comprises a substrate on which the conductors are printed.

20. (canceled)

21. A method of providing an indication of the pressure applied by a bandage to a human or animal body, the method comprising the step of positioning a disposable sensor between a bandage and the body, the sensor comprising a flat, flexible support carrying a flat, flexible pressure sensitive portion whose electrical properties vary with applied pressure perpendicular to the general plane of the support strip, and also carrying flat, flexible conductors which connect the pressure sensitive portion to a supply of electricity and to means for providing an indication of the applied pressure.

22. (canceled)

23. A pressure sensor for providing an indication of the pressure applied by a bandage to a human or animal body, the sensor comprising an flexible support substrate adapted to be placed between a bandage and the body, the support substrate carrying a flat pressure sensitive portion in the form of a flexible membrane whose electrical properties vary with applied pressure perpendicular to the plane of the membrane, and also carrying flat, flexible conductors for connecting the pressure sensitive portion to a supply of electricity and to means for providing an indication of the applied pressure.

24. A pressure sensor as claimed in claim 23, wherein the membrane has a thickness of less than 200 microns.

25. A pressure sensor for providing an indication of the pressure applied by a bandage to a human or animal body, the sensor comprising an flexible support substrate adapted to be placed between a bandage and the body, the support substrate carrying (a) a flat pressure sensitive portion whose electrical properties vary with applied pressure perpendicular to the plane of the substrate; (b) a battery; (c) means for providing an indication of the applied pressure; and (d) flexible conductors which connect the pressure sensitive portion to the supply of electricity and to the means for providing an indication of the applied pressure.

26. A method of applying a bandage to a human or animal body, in which there is provided an indication of the pressure applied by the bandage to the human or animal body and the bandage is applied in such a way that the applied pressure is kept within predetermined acceptable limits, the method comprising the step of positioning between the bandage and the body, a sensor comprising an flexible support substrate carrying (a) a flat pressure sensitive portion whose electrical properties vary with applied pressure perpendicular to the plane of the substrate; (b) a battery; (c) means for providing an indication of the applied pressure; and (d) flexible conductors which connect the pressure sensitive portion to the supply of electricity and to the means for providing an indication of the applied pressure.
